(19) United States
(12) Patent Application Publication
(10) Pub. No.: US 2012/0010131 A1
(43) Pub. Date: Jan. 12, 2012

(54) NOVEL CYCLOSPORIN ANALOGUES

(75) Inventors: Cyprian Okwara Ogbu, Durham, NC (US); Thomas Edward Richardson, Durham, NC (US)

(73) Assignee: Scynexis, Inc., Research Triangle Park, NC (US)

(21) Appl. No.: 12/981,695

(22) Filed: Dec. 30, 2010

Related U.S. Application Data
(60) Provisional application No. 61/290,917, filed on Dec. 30, 2009.

(51) Int. Cl. A61K 38/13; A61P 31/14; C12N 5/071; C07K 7/64

(52) U.S. Cl. 514/4.3; 530/321; 514/20.5; 435/375

(57) ABSTRACT

The invention relates to the use of cyclic compounds of general formula (I):

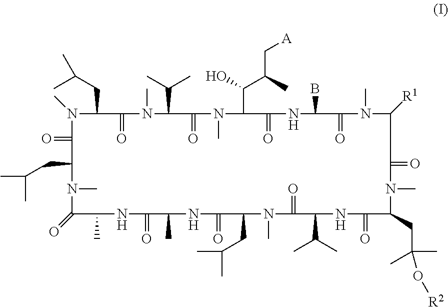

wherein A, B, R¹ and R² are as defined in the specification, and their use as pharmaceuticals.

### RELATED APPLICATIONS

This application claims the benefit of priority to U.S. Provisional Application No. 61/290,917, filed on Dec. 30, 2009, entitled “New Ether Macrocycles,” the entire contents of which is incorporated herein by reference and relied upon.

### FIELD OF THE INVENTION

This invention relates to novel compounds, compositions containing them, processes for their preparation, and their use as therapeutics, for example as antiviral agents.

### BACKGROUND OF THE INVENTION

Cyclosporine A is well known for its immunosuppressive activity and a range of therapeutic uses, including antifungal, anti-parasitic, and anti-inflammatory as well as anti-HIV activity. Cyclosporine A and certain derivatives have been reported as having anti-HCV activity, see Watashi et al., Hepatology, 2003, Vol. 38, pp 1282-1288, Nakagawa et al., Biochem. Biophys. Res. Commun. 2004, Vol. 313, pp 42-7, and Shimotohno and K. Watashi, 2004 American Transplant Congress, Abstract No. 648 (American Journal of Transplantation 2004, Volume 4, Issue s8, Pages 1-653).

Cyclosporine A (cyclosporine) derivatives modified in the 4-position to introduce hydroxyl are known in the literature. For example, [4′-Hydroxy-N-methylleucine]⁴cyclosporine A is disclosed in European Patent No. 484,281, and is stated to be active against HIV-1 replication. 3-Ether/thioether-[4′-hydroxy-N-methylleucine]⁴cyclosporine A derivatives are described in U.S. Pat. Nos. 5,948,755; 5,994,299; 5,948,884 and 6,583,265; and International Patent Publication Nos. WO2006/039668 and WO07/041631. Certain cyclosporine A derivatives with (4-acetoxy-N-methylleucine) in the 4-position and (3′-acetoxy-N-methyl-Bmt) in the 1-position are described in WO2006/039668, U.S. Pat. No. 7,196,161 B2 and Carry et al, Synlett (2004), No. 2, pages 316-320. Cyclosporine A derivatives with (4-acetoxy-N-methylleucine) in the 4-position are described in WO 98/49193, U.S. Pat. No. 5,977,067 and Carry et al, Synlett (2004), No. 2, pages 316-320. These compounds are not disclosed as having biological activity.

### SUMMARY OF THE INVENTION

In one aspect the invention provides a cyclosporine derivative of the formula (I):

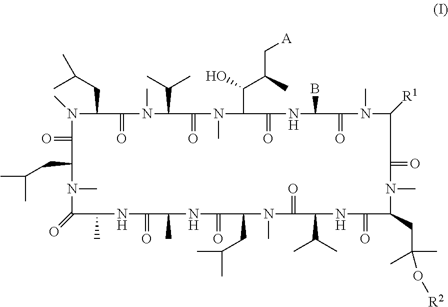

wherein:

A is (E) —CH═CHR or —CH₂CH₂R, wherein R represents methyl, —CH₂SH, —CH₂(thioalkyl), carboxyl or alkoxycarbonyl;

B represents ethyl, 1-hydroxyethyl, isopropyl or n-propyl;

R¹ represents:

hydrogen;

straight- or branched- chain alkyl having from one to six carbon atoms;

straight- or branched- chain alkenyl having from two to six carbon atoms; or —XR³;

R² represents:-

straight- or branched- chain alkyl having from one to six carbon atoms, optionally substituted by one or more groups R⁴ which are the same or different;

straight- or branched- chain alkenyl having from two to six carbon atoms optionally substituted by one or more substituents which are the same or different selected from the group consisting of halogen, hydroxy, amino, N-monoalkylamino and N,N-dialkylamino;

straight- or branched- chain alkynyl having from two to six carbon atoms, optionally substituted by one or more substituents which are the same or different selected from the group consisting of halogen, hydroxy, amino, N-monoalkylamino and N,N-dialkylamino;

or cycloalkyl having from three to six ring carbon atoms optionally substituted by one or more substituents which are the same or different selected from the group consisting of halogen, hydroxy, amino, N-monoalkylamino and N,N-dialkylamino;

X represents —S(═O)ₙ— or oxygen, where n is zero, one or two;

R³ represents:

straight- or branched- chain alkyl having from one to six carbon atoms, optionally substituted by one or more groups R⁴ which are the same or different;

straight- or branched- chain alkenyl having from two to six carbon atoms optionally substituted by one or more substituents which are the same or different selected from the group consisting of halogen, hydroxy, amino, N-monoalkylamino and N,N-dialkylamino;

straight- or branched- chain alkynyl having from two to six carbon atoms, optionally substituted by one or more substituents which are the same or different selected from the group consisting of halogen, hydroxy, amino, N-monoalkylamino and N,N-dialkylamino;

cycloalkyl having from three to six ring carbon atoms optionally substituted by one or more substituents which are the same or different selected from the group consisting of halogen, hydroxy, amino, N-monoalkylamino and N,N-dialkylamino;

or straight- or branched- chain alkoxycarbonyl having from two to six carbon atoms;

R⁴ is selected from the group consisting of halogen; hydroxy; alkoxy; carboxyl; alkoxycarbonyl; —NR⁵R⁶, —NR⁷(CH₂)ₘNR⁵R⁶; thioalkyl; phenyl optionally substituted by one or more substituents which are the same or different selected from the group consisting of halogen, alkyl, alkoxy, haloalkyl, cyano, amino, N-alkylamino and N,N-dialkylamino; and a heterocyclic ring which is saturated or unsaturated having five or six ring atoms of which from one to three heteroatoms which may be the same or different selected from nitrogen, sulfur and oxygen, wherein said heterocyclic ring is attached to (substituted onto) alkyl via a ring carbon atom;

R⁵ and R⁶, which are the same or different, each represent:

hydrogen;

straight- or branched- chain alkyl having from one to six carbon atoms, optionally substituted by one or more groups R⁷ which are the same or different; straight- or branched- chain alkenyl or alkynyl having from two to four carbon atoms;

cycloalkyl having from three to six ring carbon atoms optionally substituted by straight- or branched- chain alkyl having from one to six carbon atoms;

phenyl optionally substituted by from one to five substituents which are the same or different selected from the group consisting of halogen, alkoxy, cyano, alkoxycarbonyl, amino, alkylamino and dialkylamino;

a heterocyclic ring which is saturated or unsaturated having five or six ring atoms of which from one to three are heteroatoms which are the same or different selected from nitrogen, sulfur and oxygen, which heterocyclic ring is optionally substituted by one or more substituents which are the same or different selected from the group consisting of halogen, alkoxy, cyano, alkoxycarbonyl, amino, alkylamino and dialkylamino;

or R⁵ and R⁶, together with the nitrogen atom to which they are attached, form a saturated or unsaturated heterocyclic ring having from four to six ring atoms, which ring optionally has another ring heteroatom selected from the group consisting of nitrogen, oxygen and sulfur and is optionally substituted by from one to four substituents which are the same or different selected from the group consisting of alkyl, phenyl and benzyl;

R⁷ represents hydrogen; straight- or branched- chain alkyl having from one to six carbon atoms; or phenyl optionally substituted by from one to five substituents which are the same or different selected from the group consisting of halogen and alkoxy;

p is zero, one or two;

m is an integer from two to four;

or a pharmaceutically acceptable salt or solvate thereof

In certain cases the substituents A, B, R¹ and R² may contribute to optical and/or stereoisomerism. All such forms are embraced by the present invention.

In another aspect, provided are compositions comprising a compound provided herein along with a pharmaceutically acceptable excipient, carrier or diluent.

In another aspect, provided are pharmaceutically acceptable salts of a compound provided herein. Examples of pharmaceutically acceptable salts include salts with alkali metals, e.g., sodium, potassium or lithium, or with alkaline-earth metals, e.g., magnesium or calcium, the ammonium salt or the salts of nitrogenous bases, e.g., ethanolamine, diethanolamine, trimethylamine, triethylamine, methylamine, propylamine, diisopropylamine, N,N-dimethylethanolamine, benzylamine, dicyclohexylamine, N-benzylphenethylamine, N,N′-dibenzylethylenediamine, diphenylenediamine, benzhydrylamine, quinine, choline, arginine, lysine, leucine or dibenzylamine.

In another aspect, provided are methods of using a compound or composition provided herein to treat or prevent an infection, a neurodegenerative disease, ischemia/reperfusion damage, an inflammatory disease or an autoimmune disease. The methods generally comprise administering to a subject having the condition or disease an amount of the compound or composition effective to treat or prevent the disease or condition. Exemplary infections include HCV or HIV infection and others described in detail herein.

In another aspect, provided is a compound or composition described herein for use in therapy. In another aspect, provided is a compound or composition described herein for use in treatment or prevention of an infection, a neurodegenerative disease, ischemia/reperfusion damage, an inflammatory disease or an autoimmune disease. In another aspect, provided are uses of compounds or compositions in the manufacture of a medicament. In another aspect, provided is a compound or composition described herein for use in the manufacture of a medicament for treatment or prevention of an infection, a neurodegenerative disease, ischemia/reperfusion damage, an inflammatory disease or an autoimmune disease.

### DETAILED DESCRIPTION

Definitions

When referring to the compounds and complexes of the invention, the following terms have the following meanings unless indicated otherwise.

“Cyclosporine” refers to any cyclosporine compound known to those of skill in the art, or a derivative thereof. See, e.g., Ruegger et al., 1976, Helv. Chim. Acta. 59:1075-92; Borel et al., 1977, Immunology 32:1017-25; the contents of which are hereby incorporated by reference in their entireties. Exemplary compounds of the invention are cyclosporine derivatives. Unless noted otherwise, a cyclosporine described herein is a cyclosporine A, and a cyclosporine derivative described herein is a derivative of cyclosporine A.

The cyclosporine nomenclature and numbering systems used hereafter are those used by J. Kallen et al., “Cyclosporins: Recent Developments in Biosynthesis, Pharmacology and Biology, and Clinical Applications”, Biotechnology, second edition, H.-J. Rehm and G. Reed, ed., 1997, p535-591 and are shown below:

| Position | Amino acid in cyclosporine A |
|---|---|
| 1 | N-Methyl-butenyl-threonine (MeBmt) |
| 2 | [alpha]-aminobutyric acid (Abu) |
| 3 | Sarcosine (Sar) |
| 4 | N-Methyl-leucine (MeLeu) |
| 5 | Valine (Val) |
| 6 | N-Methyl-leucine (MeLeu) |
| 7 | Alanine (Ala) |
| 8 | (D)-Alanine ((D)-Ala) |
| 9 | N-Methyl-leucine (Me-Leu) |
| 10 | N-Methyl-leucine (MeLeu) |
| 11 | N-Methylvaline (MeVal) |

This corresponds to the saturated ring carbon atoms in the compounds of formula (I) as shown below:

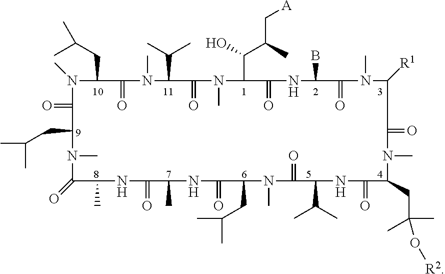

“Alkyl” refers to monovalent saturated aliphatic hydrocarbyl groups particularly having up to 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like.

“Alkylene” refers to divalent saturated aliphatic hydrocarbyl groups particularly having up to 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” refers to monovalent olefinically unsaturated hydrocarbyl groups, in one embodiment, having up to 11 carbon atoms, in another embodiment, from 2 to 8 carbon atoms, and in yet another embodiment, from 2 to 6 carbon atoms, which can be straight-chained or branched and having at least 1 or from 1 to 2 sites of olefinic unsaturation. In some embodiments, alkenyl groups include ethenyl (—CH═CH₂), n-propenyl (—CH₂CH═CH₂), isopropenyl (—C(CH₃)═CH₂), vinyl and substituted vinyl, and the like.

“Alkenylene” refers to divalent olefinically unsaturated hydrocarbyl groups particularly having up to 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically unsaturated hydrocarbyl groups particularly having up to 11 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Alkoxy” refers to the group —OR where R is alkyl. The alkyl group has up to 11 carbon atoms, more particularly as a lower alkyl, from 1 to 8 carbon atoms and still more particularly, from 1 to 6 carbon atoms. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“N-Monoalkylamino”, or “N-alkylamino” refers to the group H—NR′—, wherein R′ is selected from hydrogen and alkyl. The alkyl group has up to 11 carbon atoms, more particularly as a lower alkyl, from 1 to 8 carbon atoms and still more particularly, from 1 to 6 carbon atoms.

“Alkoxycarbonyl” refers to a radical —C(═O)-alkoxy where alkoxy is as defined herein.

“Allyl” refers to the radical H₂C═C(H)—C(H₂)—.

“Amino” refers to the radical —NH₂.

“Aryl” refers to an optionally substituted aromatic hydrocarbon radical, for example phenyl.

“Arylamino” refers to the group aryl-NR′—, wherein R′ is selected from hydrogen, aryl and heteroaryl.

“Bmt” refers to 2(S)-amino-3(R)-hydroxy-4(R)- methyl-6(E)-octenoic acid.

“Cpd” means compound.

“Carboxyl” refers to the radical —C(═O)OH.

“N,N-Dialkylamino” means a radical —NRR′ where R and R′ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

“Halogen” or “halo” refers to chloro, bromo, fluoro or iodo.

“Heteroaryl” refers to an optionally substituted saturated or unsaturated heterocyclic radical, of which from 1 to 3 are hetero ring atoms selected from the group consisting of sulfur, oxygen and nitrogen. Generally the heterocyclic ring has from 4 to 7 ring atoms, e.g. 5 or 6 ring atoms. Examples of heteroaryl include thienyl, furyl, pyrrolyl, oxazinyl, thiazinyl, pyrazinyl, pyrimidinyl, pyridazinyl, thiazolyl, oxazolyl, imidazolyl, morpholinyl, pyrazolyl, tetrahydorfuryl oxadiazolyl, thiadiazolyl and isoxazolyl.

“Hydroxy” refers to the radical —OH.

“Thioalkyl” refers to the group —SR where R is alkyl. The alkyl group has up to 11 carbon atoms, more particularly as a lower alkyl, from 1 to 8 carbon atoms and still more particularly, from 1 to 6 carbon atoms. Examples include, but are not limited to, methylthio, ethylthio, propylthio, butylthio, and the like.

“Pharmaceutically acceptable salt” refers to any salt of a compound of this invention which retains its biological properties and which is not toxic or otherwise undesirable for pharmaceutical use. Such salts may be derived from a variety of organic and inorganic counter-ions well known in the art. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Salts further include, by way of example only, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethanedisulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “physiologically acceptable cation” refers to a non-toxic, physiologically acceptable cationic counterion of an acidic functional group. Such cations are exemplified by sodium, potassium, calcium, magnesium, ammonium and tetraalkylammonium cations and the like.

“Solvate” refers to a compound of the present invention or a salt thereof that further includes a stoichiometric or non-stoichiometric amount of solvent bound by non-covalent intermolecular forces. Where the solvent is water, the solvate is a hydrate.

It is to be understood that compounds having the same molecular formula but differing in the nature or sequence of bonding of their atoms or in the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (5) according to the rules of Cahn and Prelog (Cahn et al., 1966, Angew. Chem. 78:413-447, Angew. Chem., Int. Ed. Engl. 5:385-414 (errata: Angew. Chem., Int. Ed. Engl. 5:511); Prelog and Helmchen , 1982, Angew. Chem. 94:614-631, Angew. Chem. Internat. Ed Eng. 21:567-583; Mata and Lobo, 1993, Tetrahedron; Asymmetry 4:657-668) or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof A mixture containing equal proportions of enantiomers is called a “racemic mixture”.

In certain embodiments, the compounds of this invention may possess one or more asymmetric centers; such compounds can therefore be produced as the individual (R)- or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, the present invention provides the stereoisomers of the compounds depicted herein upon treatment with base.

In certain embodiments, the compounds of the invention are “stereochemically pure”. A stereochemically pure compound has a level of stereochemical purity that would be recognized as “pure” by those of skill in the art. Of course, this level of purity will be less than 100%. In certain embodiments, “stereochemically pure” designates a compound that is substantially free of alternate isomers. In particular embodiments, the compound is 85%, 90%, 91%, 92%, 93%, 94%, 95%, 96%, 97%, 98%, 99%, 99.5% or 99.9% free of other isomers.

“Sarcosine” or “Sar” refers to the amino acid residue known to those of skill in the art having the structure —N(Me)CH₂C(═O)—. Those of skill in the art might recognize sarcosine as N-methyl glycine.

As used herein, the terms “subject” and “patient” are used interchangeably herein. The terms “subject” and “subjects” refer to an animal, in some embodiments, a mammal including a non-primate (e.g., a cow, pig, horse, cat, dog, rat, and mouse) and a primate (e.g., a monkey such as a cynomolgous monkey, a chimpanzee and a human), and a human. In another embodiment, the subject is a farm animal (e.g., a horse, a cow, a pig, etc.) or a pet (e.g., a dog or a cat). In one embodiment, the subject is a human.

As used herein, the terms “therapeutic agent” and “therapeutic agents” refer to any agent(s) which can be used in the treatment, management, or amelioration of a disorder or one or more symptoms thereof. In certain embodiments, the term “therapeutic agent” refers to a compound of the invention. In certain other embodiments, the term “therapeutic agent” does not refer to a compound of the invention. In one embodiment, a therapeutic agent is an agent that is known to be useful for, or has been or is currently being used for the treatment, management, prevention, or amelioration of a disorder or one or more symptoms thereof.

“Therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in one embodiment, to ameliorating a disease or disorder that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” refers to a compound of the invention. In certain other embodiments, the term “prophylactic agent” does not refer a compound of the invention. In one embodiment, a prophylactic agent is an agent which is known to be useful for, or has been or is currently being used to prevent or impede the onset, development, progression and/or severity of a disorder.

As used herein, the terms “prevent”, “preventing” and “prevention” refer to the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, the phrase “prophylactically effective amount” refers to the amount of a therapy (e.g., prophylactic agent) which is sufficient to result in the prevention of the development, recurrence or onset of one or more symptoms associated with a disorder, or to enhance or improve the prophylactic effect(s) of another therapy (e.g., another prophylactic agent).

The term “label” refers to a display of written, printed or graphic matter upon the immediate container of an article, for example the written material displayed on a vial containing a pharmaceutically active agent.

The term “labeling” refers to all labels and other written, printed or graphic matter upon any article or any of its containers or wrappers or accompanying such article, for example, a package insert or instructional videotapes or DVDs accompanying or associated with a container of a pharmaceutically active agent.

In certain embodiments, A represents (E) —CH═CHR. In further embodiments, A represents -CH₂CH₂R. In another embodiment, A represents (E) —CH═CHR. In a further embodiment, A is (E) —CH═CHR and R represents methyl.

In one embodiment, B represents ethyl.

In one embodiment, R¹ represents hydrogen. In another embodiment R¹ represents straight- or branched- chain alkyl having from one to six carbon atoms; straight- or branched- chain alkenyl having from two to six carbon atoms; or —XR³. In another embodiment R¹ represents —XR³. In another embodiment R¹ represents methyl.

In certain embodiments, R³ represents straight- or branched- chain alkyl having from one to six carbon atoms, In a further embodiment R³ represents methyl or ethyl. In a further embodiment R³ represents straight- or branched- chain alkyl having from one to six carbon atoms substituted by a group R⁴. In a further embodiment R³ represents straight- or branched- chain alkyl having from one to four carbon atoms substituted by a group R⁴. In a further embodiment R³ represents straight- or branched-chain alkyl having from one to four carbon atoms substituted by N,N-dialkylamino. In a further embodiment R³ represents ethyl substituted by N,N-dimethylamino.

In certain embodiments, R² represents straight- or branched- chain alkyl having from one to six carbon atoms, optionally substituted by one or more groups R⁴ which are the same or different. In a further embodiment R² represents straight- or branched- chain alkyl having from one to four carbon atoms, optionally substituted by a group R⁴. In a still further embodiment R² represents straight- or branched- chain alkyl having one or two carbon atoms, optionally substituted by a group R⁴. In a still further embodiment R² represents, methyl, ethyl, or ethyl substituted by a group R⁴.

In certain embodiments, R⁴ represents phenyl optionally substituted by one or more substituents which are the same or different selected from the group consisting of halogen, alkyl, alkoxy, haloalkyl, cyano, amino, N-alkylamino and N,N-dialkylamino. In a further embodiment R⁴ represents phenyl.

In some embodiments, X is oxygen or sulfur. In certain embodiments, X is oxygen or sulfur. In further embodiments X is oxygen. In still further embodiments, X is sulfur.

In one embodiment, the compound provided herein is selected from the following:

Cpd Name

A [4′-Benzyloxy-N-methylleucine]⁴cyclosporine A

B [4′-Ethoxy-N-methylleucine]⁴cyclosporine A

C [4′-Methoxy-N-methylleueine]⁴cyclosporine A

D [(R)-2-(N,N-Dimethylamino)ethylthio-Sar]³-[4′-ethoxy-N-methylleucine]⁴cyclosporine A.

The letters A to D are used to identify the above compounds hereafter.

The compounds of the invention can be prepared, isolated or obtained by any method apparent to those of skill in the art. Exemplary methods of preparation are described in detail in the examples below.

In one embodiment compounds of formula (I) can be prepared by the reaction of a compound of formula (II):

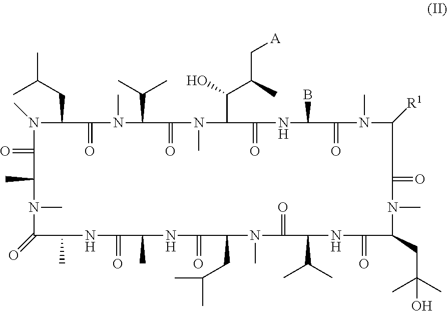

with a compound of formula CH(OR)₃ in the presence of a compound of formula R²—OH. Unexpectedly it has been found that the above reaction takes place regioselectively on the hydroxyl group at the 4-position of the cyclosporine ring, with substantially no reaction occurring on the hydroxyl group at the 1-position of the cyclosporine ring.

The reaction generally takes place a temperature of from about 0° C. to about 100° C., such as from about 0° C. to about 50° C. (for example at room temperature) in a solvent, which may be the compound of formula R²OH. The reaction may be performed in the presence of an acid, for example a Bronsted acid or a Lewis acid, or an acid such as p-toluenesulfonic acid or methanesulfonic acid. The compound of formula CH(OR²)₃ may be a trialkylorthoformate, for example triethylorthoformate.

In another embodiment compounds of formula (I) can be prepared by the reaction of a compound of formula (II) as defined above with a salt comprising a cation of formula (III):

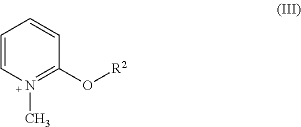

wherein R² is as defined above. Unexpectedly it has been found that the above reaction takes place regioselectively on the hydroxyl group at the 4-position of the cyclosporine ring, with substantially no reaction occurring on the hydroxyl group at the 1-position of the cyclosporine ring.

Examples of suitable salts for this reaction include salts of strong acids such as a triflate, trifluoracetate or trichloroacetate salt. The reaction is typically performed in the presence of a buffering agent such as magnesium oxide in a solvent such as an aromatic hydrocarbon, e.g. toluene or α,α,α-trifluorotoluene. The reaction is generally performed at a temperature from about 0° C. to about 100° C., such as from about 50° C. to about 100° C., e.g. from about 80° C. to about 85° C.

In another embodiment compounds of formula (I) in which R¹ represents XR³ can be prepared by treating the corresponding compound of formula (I) in which R¹ represents hydrogen with a base in an appropriate solvent to generate a polyanionic species, followed by the reaction of the polyanion thus obtained with a electrophile of formula R³X-L, wherein R³ and X are as defined above and L is a leaving group. Typically the compound of formula (I) in which R¹ is hydrogen is dissolved in an appropriate solvent and cooled to about −70° C. Solvents include tetrahydrofuran, dimethyoxymethane, methyl tert-butylether, dioxane, and the like. Following addition of a base to the mixture, the resulting mixture is generally allowed to react for about 1 hour and is optionally allowed to warm to about −20° C. The reaction mixture is typically cooled to about −70° C. and an appropriate electrophile is added. Preferred bases for this reaction include n-butyl lithium, lithium diisopropylamide, lithium diisopropylamide in combination with lithium chloride and sodium amide. Suitable electrophiles include, but are not limited to activated alkyl and alkenyl halides or sulfonates, disulfides, thiosulfonates, trialkylsilyl halides or sulfonates, and the like.

Compounds of formula (II) can be prepared according to methods known to one of skill in the art, for example, methods described in U.S. Pat. Nos. 5,948,884; 5,994,299 and 6,583,265 and in PCT publication nos. WO99/32512 and WO99/67280. The contents of these references are hereby incorporated by reference in their entireties. Compounds of formula (III) are known or can be prepared by the application and adaptation of known methods.

As discussed above, a cyclosporine compound of the invention can be in a neutral form, or in a salt form. The salt form can be any salt form known to those of skill in the art. Particularly useful salt forms are those that are coordinated with phosphate, citrate, acetate, chloride, methanesulfonate or propionate.

Where a compound of the present invention is substituted with a basic moiety, an acid addition salt can be formed. The acid which can be used to prepare an acid addition salt includes that which produces, when combined with the free base, a pharmaceutically acceptable salt, that is, a salt whose anion is non-toxic to a subject in the pharmaceutical doses of the salt. Pharmaceutically acceptable salts within the scope of the invention are those derived from the following acids: mineral acids such as hydrochloric acid, hydrobromic acid, sulfuric acid, phosphoric acid, sulfamic acid and nitric acid; and organic acids such as acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-l-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids.

The corresponding acid addition salts include hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethanedisulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

According to a further feature of the invention, acid addition salts of the compounds of this invention can be prepared by reaction of the free base with the appropriate acid, by the application or adaptation of known methods. For example, the acid addition salts of the compounds of this invention can be prepared either by dissolving the free base in aqueous or aqueous-alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds of this invention, e.g. compounds of the invention, can be regenerated from the salts by the application or adaptation of known methods. For example, parent compounds of the invention can be regenerated from their acid addition salts by treatment with an alkali, e.g., aqueous sodium bicarbonate solution or aqueous ammonia solution.

Where a compound of the invention is substituted with an acid moiety, base addition salts can be formed. Pharmaceutically acceptable salts, including for example alkali and alkaline earth metal salts, within the scope of the invention are those derived from the following bases: sodium hydride, sodium hydroxide, potassium hydroxide, calcium hydroxide, magnesium hydroxide, aluminum hydroxide, lithium hydroxide, zinc hydroxide, barium hydroxide, and organic amines such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylenediamine, chloroprocaine, diethanolamine, procaine, N-benzylphenethylamine, N-methylglucamine piperazine, tris(hydroxymethyl)-aminomethane, tetramethylammonium hydroxide, and the like.

Metal salts of compounds of the present invention can be obtained by contacting a hydride, hydroxide, carbonate or similar reactive compound of the chosen metal in an aqueous or organic solvent with the free acid form of the compound. The aqueous solvent employed may be water or it may be a mixture of water with an organic solvent, in certain embodiments, an alcohol such as methanol or ethanol, a ketone such as acetone, an aliphatic ether such as tetrahydrofuran, or an ester such as ethyl acetate. Such reactions are normally conducted at ambient temperature but they may, if desired, be conducted with heating.

Amine salts of compounds of the present invention can be obtained by contacting an amine in an aqueous or organic solvent with the free acid form of the compound. Suitable aqueous solvents include water and mixtures of water with alcohols such as methanol or ethanol, ethers such as tetrahydrofuran, nitriles, such as acetonitrile, or ketones such as acetone. Amino acid salts may be similarly prepared.

The base addition salts of the compounds of this invention can be regenerated from the salts by the application or adaptation of known methods. For example, parent compounds of the invention can be regenerated from their base addition salts by treatment with an acid, e.g., hydrochloric acid.

Pharmaceutical Compositions and Methods of Administration

The cyclosporine compounds used in the method of the present invention can be administered in certain embodiments using pharmaceutical compositions containing at least one compound of general formula (I), if appropriate in the salt form, either used alone or in the form of a combination with one or more compatible and pharmaceutically acceptable carriers, such as diluents or adjuvants, or with another anti-HCV agent. In clinical practice the cyclosporine compounds of the present invention may be administered by any conventional route, in particular orally, parenterally, rectally or by inhalation (e.g. in the form of aerosols). In one embodiment, the cyclosporine compounds of the present invention are administered orally.

Use may be made, as solid compositions for oral administration, of tablets, pills, hard gelatin capsules, powders or granules. In these compositions, the active product according to the invention is mixed with one or more inert diluents or adjuvants, such as sucrose, lactose or starch.

These compositions can comprise substances other than diluents, for example a lubricant, such as magnesium stearate, or a coating intended for controlled release

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

The compositions for parenteral administration can be emulsions or sterile solutions. Use can be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

The compositions for rectal administration are suppositories or rectal capsules which contain, in addition to the active principle, excipients such as cocoa butter, semi-synthetic glycerides or polyethylene glycols.

The compositions can also be aerosols. For use in the form of liquid aerosols, the compositions can be stable sterile solutions or solid compositions dissolved at the time of use in apyrogenic sterile water, in saline or any other pharmaceutically acceptable vehicle. For use in the form of dry aerosols intended to be directly inhaled, the active principle is finely divided and combined with a water-soluble solid diluent or vehicle, for example dextran, mannitol or lactose.

In one embodiment, a composition of the invention is a pharmaceutical composition or a single unit dosage form. Pharmaceutical compositions and single unit dosage forms of the invention comprise a prophylactically or therapeutically effective amount of one or more prophylactic or therapeutic agents (e.g., a compound of the invention, or other prophylactic or therapeutic agent), and typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In certain embodiments, water is a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions of the invention can comprise excipients that are well known in the art and are listed, for example, in the U.S. Pharmocopia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are, in certain embodiments, anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsions, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in one embodiment, in purified form, together with a suitable amount of carrier so as to provide the form for proper administration to the subject. The formulation should suit the mode of administration. In one embodiment, the pharmaceutical compositions or single unit dosage forms are sterile and in suitable form for administration to a subject, such as an animal subject, in one embodiment, a mammalian subject, such as a human subject.

A pharmaceutical composition of the invention is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil-in-water emulsions, or water-in-oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms of the invention will typically vary depending on their use. For example, a dosage form used in the initial treatment of viral infection may contain larger amounts of one or more of the active ingredients it comprises than a dosage form used in the maintenance treatment of the same infection. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 20th ed., Mack Publishing, Easton Pa. (2000).

Generally, the ingredients of compositions of the invention are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Typical dosage forms of the invention comprising a compound of the invention, or a pharmaceutically acceptable salt, solvate or hydrate thereof lie within the range of from about 0.1 mg to about 1000 mg per day, given as a single once-a-day dose in the morning or in one aspect, as divided doses throughout the day taken with food. In certain embodiments, dosage forms of the invention have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active cyclosporine.

Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pregelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC 581. Suitable anhydrous or low moisture excipients or additives include AVICEL PH 103 and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar agar, alginic acid, calcium carbonate, microcrystalline cellulose, croscarmellose sodium, crospovidone, polacrilin potassium, sodium starch glycolate, potato or tapioca starch, pregelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB O SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Delayed Release Dosage Forms

Active ingredients such as the compounds of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 each of which is incorporated herein by reference in its entirety and relied upon. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled release.

All controlled release pharmaceutical products have a common goal of improving drug therapy over that achieved by their non-controlled counterparts. Ideally, the use of an optimally designed controlled release preparation in medical treatment is characterized by a minimum of drug substance being employed to treat or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the drug may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

Parenteral Dosage Forms

Although solid, anhydrous oral dosage forms can be used, the present invention also provides parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are, in one embodiment, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Transdermal, Topical & Mucosal Dosage Forms

In one embodiment, solid, anhydrous oral dosage forms can be used. In another aspect, provided herein are transdermal, topical, and mucosal dosage forms. Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980, 1990 & 2000); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980, 1990 & 2000).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery enhancing or penetration enhancing agent. Different salts, hydrates or solvates of the active ingredients can be used to further adjust the properties of the resulting composition.

Methods of Treating or Preventing Disease in a Subject

The compounds of the present invention act on enzymes called cyclophilins and inhibit their catalytic activity and in another aspect, provided are methods to inhibit cyclophilins comprising administering a compound or composition provided herein to a subject in need thereof. Cyclophilins occur in a wide variety of different organisms, including human, yeast, bacteria, protozoa, metazoa, insects, plants, or viruses. In the case of infectious organisms, inhibition of the cyclophilin catalytic activity by compounds of the present invention often results in an inhibitory effect on the organism. Furthermore, in humans the catalytic activity of cyclophilins plays a role in many different disease situations. Inhibition of this catalytic activity is often associated to a therapeutic effect. Therefore, certain compounds of the present invention can be used for the treatment of infections including that by HCV and HIV (described further below) as well as fungal pathogens, protozoan and metazoan parasites. In addition, certain compounds of the present invention can be used to treat neurodegenerative diseases such as Alzheimer's disease, Parkinson's disease, and neuropathies. Another use of the compounds of the present invention is protection against tissue damage associated to ischemia and reperfusion such as paralytic damage after spinal cord or head injuries or cardiac damage after myocardial infarct. Furthermore, the compounds of the present invention induce regenerative processes such as that of hair, liver, gingiva, or nerve tissue damaged or lost due to injury or other underlying pathologies, such as damage of the optical nerve in glaucoma. In one embodiment the present invention provides a method inhibiting cyclophilin in a cell comprising the administration to said cell of an effective amount of a compound of formula (I) as defined above or a pharmaceutically acceptable salt or solvate thereof.

Certain compounds of the present invention can be used to treat chronic inflammatory and autoimmune diseases. As immunosuppressants, certain compounds of formula (I) are useful when administered for the prevention of immune-mediated tissue or organ graft rejection. The regulation of the immune response by the compounds of the invention would also find utility in the treatment of autoimmune diseases, such as rheumatoid arthritis, systemic lupus erythematosis, hyperimmunoglobulin E, Hashimoto's thyroiditis, multiple sclerosis, progressive systemic sclerosis, myasthenia gravis, type I diabetes, uveitis, allergic encephalomyelitis, glomerulonephritis. Further uses include the treatment and prophylaxis of inflammatory and hyperproliferative skin diseases and cutaneous manifestations of immunologically-mediated illnesses, such as psoriasis, atopic dermatitis, contact dermatitis and further eczematous dermatitises, seborrhoeic dermatitis, Lichen planus, Pemphigus, bullous pemphigoid, Epidermolysis bullosa, urticaria, angioedemas, vasculitides, erythemas, cutaneous eosinophilias, Lupus erythematosus, acne and Alopecia areata; various eye diseases (autoimmune and otherwise) such as keratoconjunctivitis, vernal conjunctivitis, keratitis, herpetic keratitis, conical cornea, dystrophia epithelialis corneae, corneal leukoma, ocular pemphigus, Mooren's ulcer, Scleritis, Graves' ophthalmopathy, Vogt-Koyanagi-Harada syndrome, sarcoidosis, multiple myeloma, etc.; obstructive airway diseases, which includes conditions such as COPD, asthma (for example, bronchial asthma, allergic asthma, intrinsic asthma, extrinsic asthma and dust asthma), particularly chronic or inveterate asthma (for example, late asthma and airway hyper-responsiveness), bronchitis, allergic rhinitis and the like; inflammation of mucosa and blood vessels such as gastric ulcers, vascular damage caused by ischemic diseases and thrombosis. Moreover, hyperproliferative vascular diseases such as intimal smooth muscle cell hyperplasia, restenosis and vascular occlusion, particularly following biologically- or mechanically-mediated vascular injury can be treated or prevented by the compounds of the invention. Other treatable conditions would include but are not limited to ischemic bowel diseases; inflammatory bowel diseases, necrotizing enterocolitis, intestinal lesions associated with thermal burns and leukotriene B4-mediated diseases; intestinal inflammations/allergies such as Coeliac diseases, proctitis, eosinophilic gastroenteritis, mastocytosis, Crohn's disease and ulcerative colitis; food-related allergic diseases which have symptomatic manifestations remote from the gastro-intestinal tract (e.g., migraine, rhinitis and eczema); renal diseases such as interstitial nephritis, Goodpasture's syndrome, hemolytic-uremic syndrome and diabetic nephropathy; nervous diseases such as multiple myositis, Guillain-Barre-syndrome, Meniere's disease, polyneuritis, multiple neuritis, mononeuritis and radiculopathy; endocrine diseases such as hyperthyroidism and Basedow's disease; hematic diseases such as pure red cell aplasia, aplastic anemia, hypoplastic anemia, idiopathic thrombocytopenic purpura, autoimmune hemolytic anemia, agranulocytosis, pernicious anemia, megaloblastic anemia and anerythroplasia; bone diseases such as osteoporosis; respiratory diseases such as sarcoidosis, fibroid lung and idiopathic interstitial pneumonia; skin disease such as dermatomyositis, leukoderma vulgaris, ichthyosis vulgaris, photoallergic sensitivity and cutaneous T cell lymphoma; circulatory diseases such as arteriosclerosis, atherosclerosis, aortitis syndrome, polyarteritis nodosa and myocardosis; collagen diseases such as scleroderma, Wegener's granuloma and Sjogren's syndrome; adiposis; eosinophilic fasciitis; periodontal disease such as lesions of gingiva, periodontium, alveolar bone and substantia ossea dentis; nephrotic syndrome such as glomerulonephritis; male pattern aleopecia or alopecia senilis by preventing epilation or providing hair germination and/or promoting hair generation and hair growth; muscular dystrophy; Pyoderma and Sezary's syndrome; Addison's disease; active oxygen-mediated diseases, as for example organ injury such as ischemia-reperfusion injury of organs (such as heart, liver, kidney and digestive tract) which occurs upon preservation, transplantation or ischemic disease (for example, thrombosis and cardiac infraction): intestinal diseases such as endotoxin-shock, pseudomembranous colitis and colitis caused by drug or radiation; renal diseases such as ischemic acute renal insufficiency and chronic renal insufficiency; pulmonary diseases such as toxinosis caused by lung-oxygen or drug (for example, paracort and bleomycins), lung cancer and pulmonary emphysema; ocular diseases such as cataracta, siderosis, retinitis pigmentosa, senile macular degeneration, vitreal scarring and corneal alkali burn; dermatitis. such as erythema multiforme, linear IgA ballous dermatitis and cement dermatitis, and others such as gingivitis, periodontitis, sepsis, pancreatitis, diseases caused by environmental pollution (for example, air pollution), aging, carcinogenis, metastasis of carcinoma and hypobaropathy; disease caused by histamine or leukotriene-C4 release; Behcet's disease such as intestinal-, vasculo- or neuro-Behcet's disease, and also Behcet's which affects the oral cavity, skin, eye, vulva, articulation, epididymis, lung, kidney and so on. Furthermore, the compounds of the invention are useful for the treatment and prevention of hepatic disease such as immunogenic diseases (for example, chronic autoimmune liver diseases such as the group consisting of autoimmune hepatitis, primary biliary cirrhosis and sclerosing cholangitis), partial liver resection, acute liver necrosis, cirrhosis (such as alcoholic cirrhosis) and hepatic failure such as fulminant hepatic failure, late-onset hepatic failure and acute liver failure or chronic liver diseases.

It will be understood that compounds of formula (I) above which possess immunosuppressive properties may not be suitable for the treatment of immunocompromised patients (e.g. patients with HIV or AIDS).

Methods of Treating or Preventing HCV and/or HIV Infection in a Subject

The present invention provides methods of using a compound or composition of the invention for the treatment or prevention of a viral infection in a subject in need thereof. The methods generally comprise the step of administering to the subject an effective amount of the compound or composition to treat or prevent the viral infection. In certain embodiments, the viral infection is HCV infection or HIV infection, or HCV and HIV co-infection.

In certain embodiments of the invention, the subject can be any subject infected with, or at risk for infection with, HCV. Infection or risk for infection can be determined according to any technique deemed suitable by the practitioner of skill in the art. In certain embodiments, subjects are humans infected with HCV. In one embodiment there is provided a method for inhibiting the replication of HCV comprising contacting HCV-infected cells with an effective amount of a compound of formula (I) as defined above, or a pharmaceutically salt or solvate thereof.

The HCV can be any HCV known to those of skill in the art. There are at least six genotypes and at least 50 subtypes of HCV currently known to those of skill in the art. The HCV can be of any genotype or subtype known to those of skill. In certain embodiments, the HCV is of a genotype or subtype not yet characterized. In certain embodiments, the subject is infected with HCV of a single genotype. In certain embodiments, the subject is infected with HCV of multiple subtypes or multiple genotypes.

In certain embodiments, the HCV is genotype 1 and can be of any subtype. For instance, in certain embodiments, the HCV is subtype 1a, 1b or 1c. It is believed that HCV infection of genotype 1 responds poorly to current interferon therapy. Methods of the present invention can be advantageous for therapy of HCV infection with genotype 1.

In certain embodiments, the HCV is other than genotype 1. In certain embodiments, the HCV is genotype 2 and can be of any subtype. For instance, in certain embodiments, the HCV is subtype 2a, 2b or 2c. In certain embodiments, the HCV is genotype 3 and can be of any subtype. For instance, in certain embodiments, the HCV is subtype 3a or 3b. In certain embodiments, the HCV is genotype 4 and can be of any subtype. For instance, in certain embodiments, the HCV is subtype 4a. In certain embodiments, the HCV is genotype 5 and can be of any subtype. For instance, in certain embodiments, the HCV is subtype 5a. In certain embodiments, the HCV is genotype 6 and can be of any subtype. For instance, in certain embodiments, the HCV is subtype 6a, 6b, 7b, 8b, 9a or 11 a. See, e.g., Simmonds, 2004, J Gen Virol. 85:3173-88; Simmonds, 2001, J. Gen. Virol., 82, 693-712, the contents of which are incorporated by reference in their entirety and relied upon.

In certain embodiments of the invention, the subject has never received therapy or prophylaxis for HCV infection. In further embodiments of the invention, the subject has previously received therapy or prophylaxis for HCV infection. For instance, in certain embodiments, the subject has not responded to HCV therapy. Indeed, under current interferon therapy, up to 50% or more HCV subjects do not respond to therapy. In certain embodiments, the subject can be a subject that received therapy but continued to suffer from viral infection or one or more symptoms thereof. In certain embodiments, the subject can be a subject that received therapy but failed to achieve a sustained virologic response. In certain embodiments, the subject has received therapy for HCV infection but has failed show a 2 log₁₀ decline in HCV RNA levels after 12 weeks of therapy. It is believed that subjects who have not shown more than 2 log₁₀ reduction in serum HCV RNA after 12 weeks of therapy have a 97-100% chance of not responding. Since the compounds of the present invention act by mechanism other than current HCV therapy, it is believed that compounds of the invention should be effective in treating such nonresponders.

In certain embodiments, the subject is a subject that discontinued HCV therapy because of one or more adverse events associated with the therapy. In certain embodiments, the subject is a subject where current therapy is not indicated. For instance, certain therapies for HCV are associated with neuropsychiatric events. Interferon (IFN)-alfa plus ribavirin is associated with a high rate of depression. Depressive symptoms have been linked to a worse outcome in a number of medical disorders. Life-threatening or fatal neuropsychiatric events, including suicide, suicidal and homicidal ideation, depression, relapse of drug addiction/overdose, and aggressive behavior have occurred in subjects with and without a previous psychiatric disorder during HCV therapy. Interferon-induced depression is a limitation for the treatment of chronic hepatitis C, especially for subjects with psychiatric disorders. Psychiatric side effects are common with interferon therapy and responsible for about 10% to 20% of discontinuations of current therapy for HCV infection.

Accordingly, the present invention provides methods of treating or preventing HCV infection in subjects where the risk of neuropsychiatric events, such as depression, contraindicates treatment with current HCV therapy. The present invention also provides methods of treating or preventing HCV infection in subjects where a neuropsychiatric event, such as depression, or risk of such indicates discontinuation of treatment with current HCV therapy. The present invention further provides methods of treating or preventing HCV infection in subjects where a neuropsychiatric event, such as depression, or risk of such indicates dose reduction of current HCV therapy.

Current therapy is also contraindicated in subjects that are hypersensitive to interferon or ribavirin, or both, or any other component of a pharmaceutical product for administration of interferon or ribavirin. Current therapy is not indicated in subjects with hemoglobinopathies (e.g., thalassemia major, sickle-cell anemia) and other subjects at risk from the hematologic side effects of current therapy. Common hematologic side effects include bone marrow suppression, neutropenia and thrombocytopenia. Furthermore, ribavirin is toxic to red blood cells and is associated with hemolysis. Accordingly, the present invention also provides methods of treating or preventing HCV infection in subjects hypersensitive to interferon or ribavirin, or both, subjects with a hemoglobinopathy, for instance thalassemia major subjects and sickle-cell anemia subjects, and other subjects at risk from the hematologic side effects of current therapy.

In certain embodiments the subject has received HCV therapy and discontinued that therapy prior to administration of a method of the invention. In further embodiments, the subject has received therapy and continues to receive that therapy along with administration of a method of the invention. The methods of the invention can be co-administered with other therapy for HCV according to the judgment of one of skill in the art. In advantageous embodiments, the methods or compositions of the invention can be co-administered with a reduced dose of the other therapy for HCV.

In certain embodiments, the present invention provides methods of treating a subject that is refractory to treatment with interferon. For instance, in some embodiments, the subject can be a subject that has failed to respond to treatment with one or more agents selected from the group consisting of interferon, interferon α, pegylated interferon α, interferon plus ribavirin, interferon α plus ribavirin and pegylated interferon α plus ribavirin. In some embodiments, the subject can be a subject that has responded poorly to treatment with one or more agents selected from the group consisting of interferon, interferon α, pegylated interferon α, interferon plus ribavirin, interferon a plus ribavirin and pegylated interferon α plus ribavirin.

In further embodiments, the present invention provides methods of treating HCV infection in subjects that are pregnant or might get pregnant since current therapy is also contraindicated in pregnant women.

In certain embodiments, the methods or compositions of the invention are administered to a subject following liver transplant. Hepatitis C is a leading cause of liver transplantation in the U.S, and many subjects that undergo liver transplantation remain HCV positive following transplantation. The present invention provides methods of treating such recurrent HCV subjects with a compound or composition of the invention. In certain embodiments, the present invention provides methods of treating a subject before, during or following liver transplant to prevent recurrent HCV infection.

Cyclosporine compounds of general formula (I) can be particularly useful in the prophylaxis and treatment of retrovirus diseases and more particularly of AIDS and of syndromes associated with AIDS. Prophylaxis is understood to mean in particular the treatment of subjects who have been exposed to HIV viruses, in particular asymptomatic seropositives who present the risk of developing the disease in the months or years to come after the primary infection. In this aspect the cyclosporine compounds of general formula (I) according to the invention can display an anti-retrovirus activity at concentrations devoid of any cytotoxic or cytostatic effect.

In embodiments of the invention, the subject can be any subject infected with, or at risk for infection with, HIV. Infection or risk for infection can be determined according to any technique deemed suitable by the practitioner of skill in the art. In certain embodiment, subjects are humans infected with HIV. The HIV can be any HIV known to those of skill in the art.

In certain embodiments of the invention, the subject has never received therapy or prophylaxis for HIV infection. In further embodiments of the invention, the subject has previously received therapy or prophylaxis for HIV infection. For instance, in certain embodiments, the subject has not responded to HIV therapy. In certain embodiments, the subject can be a subject that received therapy but continued to suffer from viral infection or one or more symptoms thereof. In certain embodiments, the subject can be a subject that received therapy but failed to achieve a sustained virologic response.

In certain embodiments, the subject is a subject that discontinued HIV therapy because of one or more adverse events associated with the therapy. In certain embodiments, the subject is a subject where current therapy is not indicated. In certain embodiments the subject has received HIV therapy and discontinued that therapy prior to administration of a method of the invention. In further embodiments, the subject has received therapy and continues to receive that therapy along with administration of a method of the invention. The methods of the invention can be co-administered with other therapy for HIV according to the judgment of one of skill in the art. In advantageous embodiments, the methods or compositions of the invention can be co-administered with a reduced dose of the other therapy for HIV.

In certain embodiments, the present invention provides methods of treating a subject that is refractory to treatment for HIV. For instance, in some embodiments, the subject can be a subject that has failed to respond to treatment with one or more therapeutic agents for HIV. In some embodiments, the subject can be a subject that has responded poorly to treatment with one or more therapeutic agents for HIV.

In certain embodiments, the subject has, or is at risk for, co-infection of HCV with HIV. For instance, in the United States, 30% of HIV subjects are co-infected with HCV and evidence indicates that people infected with HIV have a much more rapid course of their hepatitis C infection. Maier and Wu, 2002, World J Gastroenterol 8:577-57. The methods of the invention can be used to treat or prevent HCV infection in such subjects. It is believed that elimination of HCV in these subjects will lower mortality due to end-stage liver disease. Indeed, the risk of progressive liver disease is higher in subjects with severe AIDS-defining immunodeficiency than in those without. See, e.g., Lesens et al., 1999, J Infect Dis 179:1254-1258. In certain embodiments, the present invention provides methods of treating or preventing HIV infection and HCV infection in subjects in need thereof.

Dosage and Unit Dosage Forms

In human therapeutics, the doctor will determine the posology which he considers most appropriate according to a preventive or curative treatment and according to the age, weight, stage of the infection and other factors specific to the subject to be treated. Generally, doses are from about 1 to about 1500 mg per day for an adult, or from about 50 to about 1300 mg per day or from about 100 to 1100 mg per day for an adult. In one embodiment, dose rates are from about 250 to about 1000 mg per day.

In further aspects, the present invention provides methods of treating or preventing HIV and/or HCV infection in a subject by administering, to a subject in need thereof, an effective amount of a compound of the invention, or a pharmaceutically acceptable salt thereof, with a high therapeutic index against HIV and/or HCV. The therapeutic index can be measured according to any method known to those of skill in the art, such as the method described in the examples below. In certain embodiments, the therapeutic index is the ratio of a concentration at which the compound is toxic, to the concentration that is effective against HIV and/or HCV. Toxicity can be measured by any technique known to those of skill including cytotoxicity (e.g. IC₅₀ or IC₉₀) and lethal dose (e.g. LD₅₀ or LD₉₀). Likewise, effective concentrations can be measured by any technique known to those of skill including effective concentration (e.g. EC₅₀ or EC₉₀) and effective dose (e.g. ED₅₀ or ED₉₀).

The amount of the compound or composition of the invention which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions of the invention, the dosage administered to a subject is typically 0.140 mg/kg to 3 mg/kg of the subject's body weight, based on weight of the active compound. In certain aspects, the dosage administered to a subject is between 0.20 mg/kg and 2.00 mg/kg, or between 0.30 mg/kg and 1.50 mg/kg of the subject's body weight.

In general, the recommended daily dose range of a composition of the invention for the conditions described herein lie within the range of from about 0.1 mg to about 1500 mg per day, given as a single once-a-day dose or as divided doses throughout a day. In one embodiment, the daily dose is administered twice daily in equally divided doses. Specifically, a daily dose range should be from about 50 mg to about 1300 mg per day, more specifically, between about 100 mg and about 1100 mg per day, or even more specifically between about 250 and about 1000 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition of the invention are also encompassed by the above described dosage amounts and dose frequency schedules. Further, when a subject is administered multiple dosages of a composition of the invention, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of a compound or composition of the invention followed by one or more maintenance doses.

In certain embodiments, a dose of a compound or composition of the invention can be administered to achieve a steady-state concentration of the active ingredient in blood or serum of the subject. The steady-state concentration can be determined by measurement according to techniques available to those of skill or can be based on the physical characteristics of the subject such as height, weight and age.

In certain aspects, the present invention provides unit dosages comprising a compound of the invention, or a pharmaceutically acceptable salt thereof, in a form suitable for administration. Such forms are described in detail above. In certain embodiments, the unit dosage comprises 1 to 1500 mg, 5 to 250 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

Combination Therapy

The present invention provides methods of treatment or prevention that comprise the administration of a second agent effective for the treatment or prevention of HIV and/or HCV infection in a subject in need thereof. The second agent can be any agent known to those of skill in the art to be effective for the treatment or prevention of the HIV and/or HCV infection. The second agent can be a second agent presently known to those of skill in the art, or the second agent can be second agent later developed for the treatment or prevention of HIV and/or HCV. In certain embodiments, the second agent is presently approved for the treatment or prevention of HIV and/or HCV.

In certain embodiments, a compound of the invention is administered in combination with one second agent, for example a HCV agent. In further embodiments, a second agent is administered in combination with two second agents. In still further embodiments, a second agent is administered in combination with two or more second agents. Examples of a second HCV agent include interferon, pegylated interferon, ribavirin, a protease inhibitor such as telaprevir, boceprevir or ITMN-191or, or a polymerase inhibitor such as R-7128. In one embodiment a compound of the invention is provided in combination with two other HCV agents (for example pegylated interferon and ribavirin, pegylated interferon and a protease inhibitor, pegylated interferon and a polymerase inhibitor). In another aspect of this embodiment a compound of the invention is provided in combination with three other HCV agents (for example pegylated interferon, ribavirin and a protease inhibitor; pegylated interferon, ribavirin and a polymerase inhibitor; ribavirin, a protease inhibitor and a polymerase inhibitor).

Kits

The invention also provides kits for use in methods of treatment or prophylaxis of HIV and/or HCV infection. The kits can include a pharmaceutical compound or composition of the invention and instructions providing information to a health care provider regarding usage for treating or preventing a bacterial infection. Instructions may be provided in printed form or in the form of an electronic medium such as a floppy disc, CD, or DVD, or in the form of a website address where such instructions may be obtained. A unit dose of a compound or composition of the invention can include a dosage such that when administered to a subject, a therapeutically or prophylactically effective plasma level of the compound or composition can be maintained in the subject for at least 1 day. In some embodiments, a compound or composition of the invention can be included as a sterile aqueous pharmaceutical composition or dry powder (e.g., lyophilized) composition.

In some embodiments, suitable packaging is provided. As used herein, “packaging” refers to a solid matrix or material customarily used in a system and capable of holding within fixed limits a compound or composition of the invention suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

Kits of the invention may also comprise, in addition to the compound or composition of the invention, second agents or compositions comprising second agents for use with compound or composition as described in the methods above.

The following Examples illustrate the synthesis of representative cyclosporine compounds used in the present invention and the following Reference Examples illustrate the synthesis of intermediates in their preparation. These examples are not intended, nor are they to be construed, as limiting the scope of the invention. It will be clear that the invention may be practiced otherwise than as particularly described herein. Numerous modifications and variations of the present invention are possible in view of the teachings herein and, therefore, are within the scope of the invention.

### EXAMPLE 1

An oven-dried, 50 mL, round-bottomed flask is equipped with a Vigreux column with an inert gas inlet. [4′-Hydroxy-N-methylleucine]⁴cyclosporine A (250 mg, 0.205 mmol), 2-benzyloxy-l-methylpyridinium trifluoromethanesulfonate (143 mg, 0.41 mmol), magnesium oxide (light) (16.5 mg, 0.41 mmol), α,α,α-trifluorotoluene (1 mL) were added to the reaction flask. The heterogeneous reaction mixture was immersed into a 82° C. preheated oil bath and stirred for 24 hours. The reaction mixture was allowed to cool to room temperature, diluted with dichloromethane and evaporated with silica gel. Purification by flash column chromatography eluting with 0-100% of a gradient mixture of 10% methanol/ethyl acetate in heptane afforded after lyophilization [4′-benzyloxy-N-methylleucine]⁴cyclosporine A (Compound A) as a white solid. ¹H NMR (400 MHz, CDCl₃) δ ppm 0.68 (d, J=5.71 Hz, 3 H), 2.70 (s, 3 H), 2.71 (s, 3 H), 2.96 (s, 3 H), 3.09 (s, 3 H), 3.27 (s, 3 H), 3.27 (s, 3 H), 3.52 (s, 3 H), 3.74 (m, 1 H), 4.28 (d, J=10.54 Hz, 1 H), 4.34 (d, J=10.54 Hz, 1 H), 4.43 (m, 1 H),4.54 (m, 1 H), 4.61 (m, 1 H), 4.82 (m, 1 H), 4.92 (dd, J=9.08, 6.49 Hz, 1 H), 4.99 (m, 1 H), 5.06 (m, 1 H), 5.16 (d, J=10.83 Hz, 1 H), 5.35 (m, 2 H), 5.52 (m, 2 H), 5.70 (dd, J=10.69, 4.03 Hz, 1 H), 7.11 (d, J=7.91 Hz, 1 H), 7.30 (m, 5 H), 7.46 (d, J=8.15 Hz, 1 H), 7.56 (d, J=7.52 Hz, 1 H), 7.77 (d, J=9.71 Hz, 1 H); LCMS- MS (ESI+) 1308.9 (M+H).

2-Benzyloxy-l-methylpyridinium trifluoromethanesulfonate is described in Poon, K. W. C.; Albiniak, P. A.; Dudley, G. B. Org. Synth. 2007, 84, 295.

### EXAMPLE 2

Dry p-toluenesulfonic acid (127 mg, 0.74 mmol) was added to [4′-hydroxy-N-methylleucine]⁴cyclosporine A (1.0 g, 0.82 mmol) in ethanol. The flask was evacuated and refilled with argon then triethylorthoformate (2.2 mL, 14.8 mmol) was added. The mixture was stirred at room temperature for 3 days and diluted with saturated aqueous sodium hydrogen carbonate and extracted with ethyl acetate. The combined organic layers were dried, filtered and concentrated with silica gel. Purification by chromatography eluting with a gradient mixture of 0-100% ethyl acetate/heptane provided [4′-ethoxy-N-methylleucine]⁴cyclosporine A (Compound B) as a white solid. ¹H NMR (400 MHz, CDCl₃) δ ppm 0.70 (d, J=5.71 Hz, 3 H), 2.70 (s, 3 H), 2.71 (s, 3

H), 3.10 (s, 3 H), 3.13 (s, 3 H), 3.26 (s, 3 H), 3.33 (m, 2 H), 3.40 (s, 3 H), 3.52 (s, 3 H), 3.78 (m, 1 H), 4.54 (m, 1 H), 4.63 (t, J=9.12 Hz, 1 H), 4.70 (m, 1 H), 4.83 (m, 1 H), 4.95 (m, 1 H), 5.06 (m, 2 H), 5.15 (d, J=10.64 Hz, 1 H), 5.35 (m, 2 H), 5.51 (m, 2 H), 5.72 (dd, J=10.88, 4.15 Hz, 1 H), 7.14 (d, J=7.91 Hz, 1 H), 7.49 (d, J=8.15 Hz, 1 H), 7.59 (d, J=7.52 Hz, 1 H), 7.87 (d, J=9.76 Hz, 1 H); LCMS- MS (ESI+) 1246.9 (M+H);

and [4′-dehydro]⁴cyclosporine A ¹H NMR (400 MHz, CDC1₃) δ ppm 0.73 (d, J=6.10 Hz, 3 H), 2.70 (s, 3 H), 2.71 (s, 3 H), 3.10 (s, 3 H), 3.12 (s, 3 H), 3.27 (s, 3 H), 3.40 (s, 3 H), 3.51 (s, 3 H), 3.79 (m, 1 H), 4.53 (m, 1 H), 4.68 (t, J=8.80 Hz, 1 H), 4.71 (br s, 1 H), 4.77 (br s, 1 H), 4.82 (m, 1 H), 4.99 (m, 1 H), 5.06 (m, 2 H), 5.13 (d, J=10.83 Hz, 1 H), 5.35 (m, 2 H), 5.49 (d, J=4.00 Hz, 1 H), 5.52 (dd, J=12.10, 3.37 Hz, 1 H), 5.71 (dd, J=10.98, 3.90 Hz, 1 H), 7.17 (d, J=7.96 Hz, 1 H), 7.53 (d, J=8.35 Hz, 1 H), 7.65 (d, J=7.42 Hz, 1 H), 8.00 (d, J=9.81 Hz, 1 H); LCMS- MS (ESI+) 1200.9 (M+H).

By proceeding in a similar manner, replacing ethanol with methanol and triethylorthoformate with trimethylorthoformate, [4′-methoxy-N-methylleucine]⁴cyclosporine A (Compound C) was prepared, ¹H NMR (400 MHz, CDCl₃) δ ppm 0.70 (d, J=5.71 Hz, 3 H), 2.70 (s, 3 H), 2.71 (s, 3 H), 3.11 (s, 3 H), 3.13 (s, 3 H), 3.26 (s, 3 H), 3.40 (s, 3 H), 3.52 (s, 3 H), 3.78 (m, 1 H), 4.12 (m, 1 H), 4.54 (m, 1 H), 4.64 (t, J=9.12 Hz, 1 H), 4.70 (m, 1 H), 4.83 (m, 1 H), 4.95 (m, 1 H), 5.07 (m, 2 H), 5.15 (d, J=10.88 Hz, 1 H), 5.35 (m, 2 H), 5.50 (m, 2 H), 5.72 (dd, J=10.79, 4.03 Hz, 1 H), 7.13 (d, J=7.91 Hz, 1 H), 7.49 (d, J=8.15 Hz, 1 H), 7.59 (d, J=7.52 Hz, 1 H), 7.87 (d, J=9.81 Hz, 1 H); LCMS- MS (ESI+) 1232.2 (M+H). [4′-Dehydro]⁴cyclosporine A was obtained as a second product of the reaction.

### EXAMPLE 3

p-Toluene sulfonic acid monohydrate (12.2 mg, 0.064 mmol) was added to [(R)-2-(N,N-dimethylamino)ethylthio-Sar]³-[4′-hydroxy-N-methylleucine]⁴cyclosporine A (51.5 mg, 0.039 mmol) in 2 mL of ethanol. The flask was evacuated and refilled with argon then triethylorthoformate (0.116 mL, 0.701 mmol) was added. The mixture was stirred at room temperature for 3 days and evaporated with silica gel. Purification by chromatography eluting with a gradient mixture of 0-100% dichloromethane/methanol/ammonium hydroxide in dichloromethane provided [(R)-2-(N,N-dimethylamino)ethylthio-Sail³44′-ethoxy-N-methylleucine]⁴cyclosporine A (Compound D) as a white solid, ¹H NMR (400 MHz, CDCl₃) δ ppm 0.69 (d, J=6.10 Hz, 3 H), 2.24 (s, 6 H), 2.70 (s, 6 H), 3.12 (s, 3 H), 3.16 (s, 3 H), 3.26 (s, 3 H), 3.35 (m, 2 H), 3.45 (s, 3 H), 3.49 (s, 3 H), 3.76 (m, 1 H), 4.54 (m, 1 H), 4.64 (t, J=9.13 Hz, 1 H), 4.84 (m, 1 H), 4.98 (m, 1 H), 5.07 (m, 2 H), 5.14 (d, J=10.79 Hz, 1 H), 5.34 (m, 2 H), 5.51 (dd, J=15.91, 6.30 Hz, 1 H), 5.70 (dd, J=10.59, 3.76 Hz, 1 H), 6.02 (s, 1 H), 7.14 (d, J=8.35 Hz, 1 H), 7.37 (d, J=8.15 Hz, 1 H), 7.63 (d, J=7.52 Hz, 1 H), 7.95 (d, J=9.71 Hz, 1 H); LCMS- MS (ESI+) 1349.7 (M+H);

and [(R)-2-(N,N-dimethylamino)ethylthio-Sar]³-[4′-dehydro-N-methylleucine]⁴cyclosporine A ¹H NMR (400 MHz, CDCl₃) δ ppm 0.71 (d, J=5.91 Hz, 3 H), 2.24 (s, 6 H), 2.70 (s, 6 H), 3.11 (s, 3 1-1), 3.12 (s, 3 H), 3.27 (s, 3 H), 3.43 (s, 3 H), 3.51 (s, 3 H), 3.86 (m, 1 H), 4.55 (m, 1 H), 4.68 (t, J=8.80 Hz, 1 H), 4.71 (br s, 1 H), 4.77 (br s, 1 H), 4.82 (m, 1 H), 4.99 (m, 1 H), 5.06 (m, 2 1-1), 5.13 (d, J=10.83 Hz, 1 H), 5.35 (m, 2 H), 5.49 (d, J=4.00 Hz, 1 H), 5.52 (dd, J=12.10, 3.37 Hz, 1 H), 5.71 (dd, J=10.98, 3.90 Hz, 1 H), 5.95 (s, 1 H), 7.17 (d, J=7.96 Hz, 1 H), 7.53 (d, J=8.35 Hz, 1 H), 7.65 (d, J=7.42 Hz, 1 H), 8.00 (d, J=9.81 Hz, 1 H); LCMS- MS (ESI+) 1303.6 (M+H).

Etherification with triethylorthoformate is described in Linnanen et al, Journal of Medicinal.Chemistry (2000) Volume 43, page 1339.

[4′-Hydroxy-N-methylleucine]⁴cyclosporine A was prepared according to the method described in European Patent No. 484,281, the disclosure of which is specifically incorporated by reference its entiretyand relied upon.

HCV Activity

The compounds of the present invention were tested for activity against HCV using the methods adapted from those described by Kriger et al, Journal of Virology, 2001 volume 75, p. 4614-4624, Pietschmann et al, Journal of Virology, 2002 volume 76, p. 4008-4021, and using HCV RNA constructs as described in U.S. Pat. No. 6,630,343, both incorporated by reference herein in their entireties and relied upon. Compounds were examined in the human hepatoma cell line ET (lub ubi neo/ET), a HCV RNA replicon containing a stable luciferase (LUC) reporter. The HCV RNA replicon ET contains the 5′ end of HCV (with the HCV Internal Ribosome Entry Site (IRES) and the first few amino acids of the HCV core protein) which drives the production of a firefly luciferase (LUC), ubiquitin, and neomycin phosphotransferase (NeoR) fusion protein. Ubiquitin cleavage releases the LUC and NeoR proteins. The EMCV IRES element controls the translation of the HCV structural proteins NS3-NS5. The NS3 protein cleaves the HCV polyprotein to release the mature NS3, NS4A, NS4B, NS5A and NSSB proteins that are required for HCV replication. At the 3′ end of the replicon is the authentic 3′ NTR of HCV. The activity of the LUC reporter is directly proportional to HCV replication levels and positive-control antiviral compounds produce a reproducible antiviral response using the LUC endpoint.

The compounds are dissolved in DMSO at five half-log concentrations each, ranging from either 0.03 to 3 μM or 1 to 100 μM. Subconfluent cultures of the ET line are plated out into 96 well plates dedicated for the analysis of cell numbers (cytotoxicity) or antiviral activity and the next day the compounds are added to the appropriate wells. The cells are processed 72 hours later when the cells were still subconfluent. Antiviral activity is expressed as EC₅₀ and EC₉₀, the effective concentration of compound that reduced viral replication by 50% and 90%, respectively. Compound EC₅₀ and EC₉₀ values are derived from HCV RNA levels assessed as HCV RNA replicon derived LUC activity. Cytotoxicity is expressed as IC₅₀ and 1C₉₀, the concentration of compound that inhibit cell viability by 50% and 90%, respectively. Compound IC₅₀ and IC₉₀ values are calculated using a colorimetric assay as an indication of cell numbers and cytotoxicity. The activity of the LUC reporter is directly proportional to HCV RNA levels in the human cell line. The HCV-replicon assay is validated in parallel experiments using interferon-alpha-2b as a positive control. Cyclosporine A is tested by way of comparison. Representative compounds of the invention demonstrated activity in this assay. By way of example, compound A gave an EC₅₀ value of 410 nM.

HIV Activity

The compounds of the present invention are also tested for antiretroviral activity against human immunodeficiency virus-1 (HIV) using infection of the human T-lymphoblastoid cell line, CEM-SS, with the HIV strain HIV-1IIIB (Weislow et al., 1989, J. Natl. Cancer Inst. 81:577-586). In this MTS cytoprotection assay, each experiment includes cell control wells (cells only), virus control wells (cells plus virus), drug toxicity wells (cells plus drug only), drug colorimetric control wells (drug only) as well as experimental wells (drug plus cells plus virus). Compounds are first dissolved in DMSO and tested using six half-log dilutions, starting with a high concentration of either 20 or 2 μM. HIV-1RF is added to each well in a volume of 50 !μL, the amount of virus determined to give approximately 90% cell killing at 6 days post-infection. At assay termination, assay plates are stained with the soluble tetrazolium-based dye MTS (CellTiter 96 Reagent, Promega) to determine cell viability and quantify compound toxicity. MTS is metabolized by the mitochondria enzymes of metabolically active cells to yield a soluble formazan product, providing a quantitative analysis of cell viability and compound cytotoxicity. The assay is validated in parallel experiments using Zidovudine (3′-azido-3′-deoxythymidine or AZT) as a positive control. The assay includes determinations of compound EC₅₀ (concentration inhibiting virus replication by 50%), IC₅₀ (concentration resulting in 50% inhibition of cell growth) and a selectivity index (IC₅₀/EC₅₀).

Cyclophilin Binding Activity

The cyclophilin inhibition binding of compounds of the invention was determined using a competitive ELISA adapted from the methods described by Quesniaux et al. (Eur. J Immunol. 1987, Vol. 17, pages 1359-1365). Activated ester of succinyl spacers bound to D-Lys⁸-cylosporine A (D-Lys⁸-Cs) was coupled to bovine serum albumin (BSA) through D-lysyl residue in position 8. BSA was dissolved in 0.1 M borate buffer, pH 9.0 (4 mg in 1.4 ml). A hundredfold molar excess of D-Lys⁸-Cs dissolved in dimethyl formamide (0.6 ml) was added dropwise to the BSA under vigorous stirring. The coupling reaction was performed for 2 to 3 hours at room temperature under mild stirring and the conjugate was extensively dialyzed against phosphate-buffered saline (PBS, pH 7.4). After acetone precipitation of an aliquot of the conjugated protein, no covalently bound D-Lys⁸-Cs remained in the acetone solution and the extent of cyclosporine covalent binding was calculated.

Microtiter Plates were coated with D-Lys⁸-Cs-BSA conjugate (2 pg/ml in PBS for 24 hours at 4° C.). Plates were washed with Tween®/PBS and with PBS alone. To block nonspecific binding, 2% BSA/PBS (pH 7.4) was added to the wells and allowed to incubate for 2 hours at 37° C. A five-fold dilution series of the compound to be tested was made in ethanol in a separate microtiter plate. The starting concentration was 0.1 mg/mL for assays with human recombinant cyclophilin. 198 μL of 0.1 μg/mL cyclophilin solution was added to the microtiter immediately followed by 2 μL of diluted cyclosporine A (used as a reference compound) or the compound of the invention. The reaction between coated BSA-Cs conjugate, free cyclosporine A and cyclophilin was allowed to equilibrate overnight at 4° C. Cyclophilin was detected with anti-cyclophilin rabbit antiserum diluted in 1% BSA containing PBS and incubated overnight at 4° C. Plates were washed as described above. Bound rabbit antibodies were then detected by goat anti-rabbit IgG conjugated to alkaline phosphatase diluted in 1% BSA-PBS and allowed to incubate for 2 hours at 37° C. Plates were washed as described above. After incubation with 4-nitrophenyl phosphate (1 g/1 in diethanolamine buffer, pH 9.8) for 1 to 2 hours at 37° C., the enzymatic reaction was measured spectrophotometrically at 405 nm using a spectrophotometer.

The results were as follows: Compound A had a cyclophilin A binding value of 98 ng/mL or lower and a cyclophilin B binding value of 102 ng/ml and a cyclophilin D binding value of 124 ng/ml, illustrating the ability of representative compounds of the invention to inhibit cyclophilin.

Compounds of the invention were tested for their T Cell stimulation (IL-2) in Jurkat cells with anti-CD3 and anti-CD28 co-stimulation. All compounds had a 0.5-Log 9-point titration starting at 10 μM (n=2) to 0.0015 μM . Cyclosporine A (control) was also run at a 0.5-Log 9-point titration, starting at 500 ng/mL. All compounds to be tested were dissolved in dimethyl sulfoxide. Cytotoxicity was evaluated with parallel Alamar Blue plates. Jurkat cells were seeded at 2×10⁵ cells per well in 190 μL growth media in a 96-well plate, Cells were cultured in RPMI 1640 medium, 10% fetal bovine serum, and L-Glutamine with incubation at 37° C. with 5% carbon dioxide. After 1 hour of incubation the cells were stimulated with immobilized anti-CD3 (0.4m/well), anti-CD28 soluble (2 μg/mL). After 6 hours the sample supernatants were harvested and stored at −80° C. 50 μL samples of supernatant were tested for IL-2 using a Luminex® 1-plex assay.

The following representative IL-2 activity results were obtained: The EC₅₀ value for Compound A was 43 ng/ml, in the absence of cytotoxicity.

Mitochondrial permeability transition

Mitochondrial permeability transition (MPT) was determined by measuring swelling of the mitochondria induced by Ca²⁺. The procedure was adapted from the method described by Blattner et al, Analytical Biochem, Volume 295, page 220 (2001). Mitochondria were prepared from rat livers, which had been perfused with phosphate-buffered saline (PBS) to remove blood, using standard methods that utilized gentle homogenization in sucrose based buffer and then differential centrifugation to first remove cellular debris and then to pellet the mitochondria. Swelling was induced by 150 micromolar Ca²⁺ (added from a concentrated solution of calcium chloride) and was monitored by measuring the scattering at 535-540 nm. Representative compounds were added 5 minutes before swelling was induced. EC₅₀ were determined by comparing swelling with and without the compounds of the invention.

In the above test Compound A gave an EC₅₀ value less than of 10 μM, indicating the ability of representative compounds of the invention to penetrate mitochondria and inhibit the MPT.

All publications and patent applications cited in this specification are herein incorporated by reference in their entireties as if each individual publication or patent application were specifically and individually indicated to be incorporated by reference. While the invention has been described in terms of various embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A compound of the formula (I): 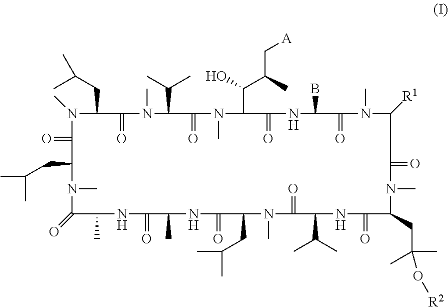
wherein:
A is (E) —CH═CHR or —CH₂CH₂R, wherein R represents methyl, —CH₂SH, —CH₂(thioalkyl), carboxyl or alkoxycarbonyl;
B represents ethyl, 1-hydroxyethyl, isopropyl or n-propyl;
R¹ represents:
hydrogen;
straight- or branched- chain alkyl having from one to six carbon atoms;
straight- or branched- chain alkenyl having from two to six carbon atoms;
or —XR³;
R² represents:-
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
or cycloalkyl having from three to six ring carbon atoms optionally substituted by one or more substituents which are the same or different selected from the group consisting of halogen, hydroxy, amino, N-monoalkylamino and N,N-dialkylamino;
X represents —S(═O)ₙ— or oxygen, where n is zero, one or two;
R³ represents:
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
cycloalkyl having from three to six ring carbon atoms optionally substituted by one or more substituents which are the same or different selected from the group consisting of halogen, hydroxy, amino, N-monoalkylamino and N,N-dialkylamino;
or straight- or branched- chain alkoxycarbonyl having from two to six carbon atoms;
R⁴ is selected from the group consisting of halogen; hydroxy; alkoxy; carboxyl;
alkoxycarbonyl; —NR⁵R⁶; —NR⁷(CH₂)ₘNR⁵R⁶; thioalkyl; phenyl optionally substituted by one or more substituents which are the same or different selected from the group consisting of halogen, alkyl, alkoxy, haloalkyl, cyano, amino, N-alkylamino and N,N-dialkylamino; and a heterocyclic ring which is saturated or unsaturated having five or six ring atoms of which from one to three are heteroatoms which are the same or different selected from nitrogen, sulfur and oxygen, wherein said heterocyclic ring is attached to (substituted onto) alkyl via a ring carbon atom;
R⁵ and R⁶, which are the same or different, each represent:
hydrogen;
straight- or branched- chain alkyl having from one to six carbon atoms, optionally substituted by one or more groups R⁷ which are the same or different;
straight- or branched- chain alkenyl or alkynyl having from two to four carbon atoms;
cycloalkyl having from three to six ring carbon atoms optionally substituted by straight- or branched- chain alkyl having from one to six carbon atoms;
phenyl optionally substituted by from one to five substituents which are the same or different selected from the group consisting of halogen, alkoxy, cyano, alkoxycarbonyl, amino, alkylamino and dialkylamino;
a heterocyclic ring which is saturated or unsaturated having five or six ring atoms of which from one to three are heteroatoms which are same or different selected from nitrogen, sulfur and oxygen, which heterocyclic ring is optionally substituted by one or more substituents which are the same or different selected from the group consisting of halogen, alkoxy, cyano, alkoxycarbonyl, amino, alkylamino and dialkylamino;
or R⁵ and R⁶, together with the nitrogen atom to which they are attached, form a saturated or unsaturated heterocyclic ring having from four to six ring atoms, which ring optionally has another ring heteroatom selected from the group consisting of nitrogen, oxygen and sulfur and is optionally substituted by from one to four substituents which are the same or different selected from the group consisting of alkyl, phenyl and benzyl;
R⁷ represents hydrogen; straight- or branched- chain alkyl having from one to six carbon atoms; or phenyl optionally substituted by from one to five substituents which are the same or different selected from the group consisting of halogen and alkoxy;
p is zero, one or two;
m is an integer from two to four;
or a pharmaceutically acceptable salt or solvate thereof.

2. The compound according to claim 1 in which A represents (E) —CH═CHR, R represents methyl and B represents ethyl.

3. The compound according to claim 1 in which R¹ represents hydrogen.

4. The compound according to claim 1 in which R² represents straight- or branched- chain alkyl having from one to six carbon atoms, optionally substituted by one or more groups R⁴ which are the same or different, wherein R⁴ is as defined in claim 1.

5. The compound according to claim 4 in which R⁴ represents phenyl optionally substituted by one or more substituents which are the same or different selected from the group consisting of halogen, alkyl, alkoxy, haloalkyl, cyano, amino, N-alkylamino and N,N-dialkylamino.

6. The compound according to claim 1 which is selected from the group consisting of [4′-benzyloxy-N-methylleucine]⁴cyclosporine A; [4′-ethoxy-N-methylleucine]⁴cyclosporine A; and [4′-methoxy-N-methylleucine]⁴cyclosporine A.

7. A compound of the formula (I): 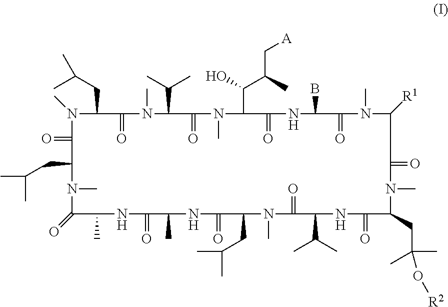
wherein:
A is (E) —CH═CHR or —CH₂CH₂R, wherein R represents methyl, —CH₂SH, —CH₂(thioalkyl), carboxyl or alkoxycarbonyl;
B represents ethyl, 1-hydroxyethyl, isopropyl or n-propyl;
R¹ represents:
straight- or branched- chain alkyl having from one to six carbon atoms;
straight- or branched- chain alkenyl having from two to six carbon atoms;
or —XR³;
R² represents:-
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
or cycloalkyl having from three to six ring carbon atoms optionally substituted by one or more substituents which are the same or different selected from the group consisting of halogen, hydroxy, amino, N-monoalkylamino and N,N-dialkylamino;
X represents —S(═O)ₙ— or oxygen, where n is zero, one or two;
R³ represents:
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
cycloalkyl having from three to six ring carbon atoms optionally substituted by one or more substituents which are the same or different selected from the group consisting of halogen, hydroxy, amino, N-monoalkylamino and N,N-dialkylamino;
or straight- or branched- chain alkoxycarbonyl having from two to six carbon atoms;
R⁴ is selected from the group consisting of halogen; hydroxy; alkoxy; carboxyl;
alkoxycarbonyl; —NR⁵R⁶; —NR⁷(CH₂)ₘNR⁵R⁶; thioalkyl; phenyl optionally substituted by one or more substituents which are the same or different selected from the group consisting of halogen, alkyl, alkoxy, haloalkyl, cyano, amino, N-alkylamino and N,N-dialkylamino; and a heterocyclic ring which is saturated or unsaturated having five or six ring atoms of which from one to three are heteroatoms which are the same or different selected from nitrogen, sulfur and oxygen, wherein said heterocyclic ring is attached to (substituted onto) alkyl via a ring carbon atom;
R⁵ and R⁶, which are the same or different, each represent:
hydrogen;
straight- or branched- chain alkyl having from one to six carbon atoms, optionally substituted by one or more groups R⁷ which are the same or different;
straight- or branched- chain alkenyl or alkynyl having from two to four carbon atoms;
cycloalkyl having from three to six ring carbon atoms optionally substituted by straight- or branched- chain alkyl having from one to six carbon atoms;
phenyl optionally substituted by from one to five substituents which are the same or different selected from the group consisting of halogen, alkoxy, cyano, alkoxycarbonyl, amino, alkylamino and dialkylamino;
a heterocyclic ring which is saturated or unsaturated having five or six ring atoms of which from one to three are heteroatoms which are same or different selected from nitrogen, sulfur and oxygen, which heterocyclic ring is optionally substituted by one or more substituents which are the same or different selected from the group consisting of halogen, alkoxy, cyano, alkoxycarbonyl, amino, alkylamino and dialkylamino;
or R⁵ and R⁶, together with the nitrogen atom to which they are attached, form a saturated or unsaturated heterocyclic ring having from four to six ring atoms, which ring optionally has another ring heteroatom selected from the group consisting of nitrogen, oxygen and sulfur and is optionally substituted by from one to four substituents which are the same or different selected from the group consisting of alkyl, phenyl and benzyl;
R⁷ represents hydrogen; straight- or branched- chain alkyl having from one to six carbon atoms; or phenyl optionally substituted by from one to five substituents which are the same or different selected from the group consisting of halogen and alkoxy;
p is zero, one or two;
m is an integer from two to four;
or a pharmaceutically acceptable salt or solvate thereof.

8. The compound according to claim 7 in which R¹ represents —XR³.

9. The compound according to claim 8 in which X represents sulfur.

10. The compound according to claim 8 in which X represents oxygen.

11. The compound according to claim 7 in which R¹ represents methyl.

12. The compound according to claim 7 in which R² represents straight- or branched- chain alkyl having from one to six carbon atoms, optionally substituted by one or more groups R⁴ which are the same or different, wherein R⁴ is as defined in claim 7.

13. The compound according to claim 12 in which R⁴ represents phenyl optionally substituted by one or more substituents which are the same or different selected from the group consisting of halogen, alkyl, alkoxy, haloalkyl, cyano, amino, N-alkylamino and N,N-dialkylamino.

14. The compound according to claim 7 which is [(R)-2-(N,N-dimethylamino)-ethylthio-Sar]³-[4′-ethoxy-N-methylleucine]⁴cyclosporine A.

15. A composition comprising a compound of the formula (I): 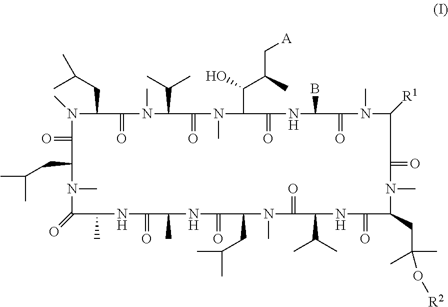
wherein:
A is (E) —CH═CHR or —CH₂CH₂R, wherein R represents methyl, —CH₂SH, —CH₂(thioalkyl), carboxyl or alkoxycarbonyl;
B represents ethyl, 1-hydroxyethyl, isopropyl or n-propyl;
R¹ represents:
hydrogen;
straight- or branched- chain alkyl having from one to six carbon atoms;
straight- or branched- chain alkenyl having from two to six carbon atoms;
or —XR³;
R² represents:-
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
or cycloalkyl having from three to six ring carbon atoms optionally substituted by one or more substituents which are the same or different selected from the group consisting of halogen, hydroxy, amino, N-monoalkylamino and N,N-dialkylamino;
X represents —S(═O)ₙ— or oxygen, where n is zero, one or two;
R³ represents:
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
cycloalkyl having from three to six ring carbon atoms optionally substituted by one or more substituents which are the same or different selected from the group consisting of halogen, hydroxy, amino, N-monoalkylamino and N,N-dialkylamino;
or straight- or branched- chain alkoxycarbonyl having from two to six carbon atoms;
R⁴ is selected from the group consisting of halogen; hydroxy; alkoxy; carboxyl;
alkoxycarbonyl; —NR⁵R⁶; —NR⁷(CH₂)ₘ,NR⁵R⁶; thioalkyl; phenyl optionally substituted by one or more substituents which are the same or different selected from the group consisting of halogen, alkyl, alkoxy, haloalkyl, cyano, amino, N-alkylamino and N,N-dialkylamino; and a heterocyclic ring which is saturated or unsaturated having five or six ring atoms of which from one to three are heteroatoms which are the same or different selected from nitrogen, sulfur and oxygen, wherein said heterocyclic ring is attached to (substituted onto) alkyl via a ring carbon atom;
R⁵ and R⁶, which are the same or different, each represent:
hydrogen;
straight- or branched- chain alkyl having from one to six carbon atoms, optionally substituted by one or more groups R⁷ which are the same or different;
straight- or branched- chain alkenyl or alkynyl having from two to four carbon atoms;
cycloalkyl having from three to six ring carbon atoms optionally substituted by straight- or branched- chain alkyl having from one to six carbon atoms;
phenyl optionally substituted by from one to five substituents which are the same or different selected from the group consisting of halogen, alkoxy, cyano, alkoxycarbonyl, amino, alkylamino and dialkylamino;
a heterocyclic ring which is saturated or unsaturated having five or six ring atoms of which from one to three are heteroatoms which are same or different selected from nitrogen, sulfur and oxygen, which heterocyclic ring is optionally substituted by one or more substituents which are the same or different selected from the group consisting of halogen, alkoxy, cyano, alkoxycarbonyl, amino, alkylamino and dialkylamino;
or R⁵ and R⁶, together with the nitrogen atom to which they are attached, form a saturated or unsaturated heterocyclic ring having from four to six ring atoms, which ring optionally has another ring heteroatom selected from the group consisting of nitrogen, oxygen and sulfur and is optionally substituted by from one to four substituents which are the same or different selected from the group consisting of alkyl, phenyl and benzyl;
R⁷ represents hydrogen; straight- or branched- chain alkyl having from one to six carbon atoms; or phenyl optionally substituted by from one to five substituents which are the same or different selected from the group consisting of halogen and alkoxy;
p is zero, one or two;
m is an integer from two to four;
or a pharmaceutically acceptable salt or solvate thereof, and a pharmaceutically acceptable excipient, carrier or diluent.

16. A composition comprising a compound of the formula (I) as defined in claim 7 and a pharmaceutically acceptable excipient, carrier or diluent.

17. A method for treating or preventing HCV in a patient in need of same comprising administering to said patient an effective anti-HCV amount of a compound of the formula (I): 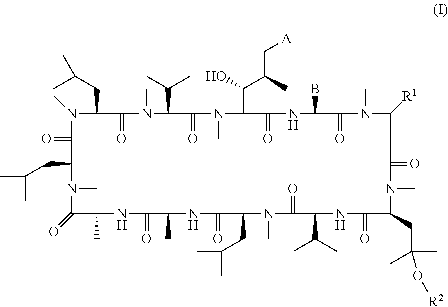
wherein:
A is (E) —CH═CHR or —CH₂CH₂R, wherein R represents methyl, —CH₂SH, —CH₂(thioalkyl), carboxyl or alkoxycarbonyl;
B represents ethyl, 1-hydroxyethyl, isopropyl or n-propyl;
R¹ represents:
hydrogen;
straight- or branched- chain alkyl having from one to six carbon atoms;
straight- or branched- chain alkenyl having from two to six carbon atoms;
or —XR³;
R² represents:-
straight- or branched- chain alkyl having from one to six carbon atoms, optionally
substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
or cycloalkyl having from three to six ring carbon atoms optionally substituted by one or more substituents which are the same or different selected from the group consisting of halogen, hydroxy, amino, N-monoalkylamino and N,N-dialkylamino;
X represents —S(═O)ₙ— or oxygen, where n is zero, one or two;
R³ represents:
straight- or branched- chain alkyl having from one to six carbon atoms, optionally substituted by one or more groups R⁴ which are the same or different;
straight- or branched- chain alkenyl having from two to six carbon atoms optionally substituted by one or more substituents which are the same or different selected from the group consisting of halogen, hydroxy, amino, N-monoalkylamino and N,N-dialkylamino;
straight- or branched- chain alkynyl having from two to six carbon atoms, optionally substituted by one or more substituents which are the same or different selected from the group consisting of halogen, hydroxy, amino, N-monoalkylamino and N,N-dialkylamino;
cycloalkyl having from three to six ring carbon atoms optionally substituted by one or more substituents which are the same or different selected from the group consisting of halogen, hydroxy, amino, N-monoalkylamino and N,N-dialkylamino;
or straight- or branched- chain alkoxycarbonyl having from two to six carbon atoms;
R⁴ is selected from the group consisting of halogen; hydroxy; alkoxy; carboxyl;
alkoxycarbonyl; —NR⁵R⁶; —NR⁷(CH₂)ₘNR⁵R⁶; thioalkyl; phenyl optionally substituted by one or more substituents which are the same or different selected from the group consisting of halogen, alkyl, alkoxy, haloalkyl, cyano, amino, N-alkylamino and N,N-dialkylamino; and a heterocyclic ring which is saturated or unsaturated having five or six ring atoms of which from one to three are heteroatoms which are the same or different selected from nitrogen, sulfur and oxygen, wherein said heterocyclic ring is attached to (substituted onto) alkyl via a ring carbon atom;
R⁵ and R⁶, which are the same or different, each represent:
hydrogen;
straight- or branched- chain alkyl having from one to six carbon atoms, optionally substituted by one or more groups R⁷ which are the same or different;
straight- or branched- chain alkenyl or alkynyl having from two to four carbon atoms;
cycloalkyl having from three to six ring carbon atoms optionally substituted by straight- or branched- chain alkyl having from one to six carbon atoms;
phenyl optionally substituted by from one to five substituents which are the same or different selected from the group consisting of halogen, alkoxy, cyano, alkoxycarbonyl, amino, alkylamino and dialkylamino;
a heterocyclic ring which is saturated or unsaturated having five or six ring atoms of which from one to three are heteroatoms which are same or different selected from nitrogen, sulfur and oxygen, which heterocyclic ring is optionally substituted by one or more substituents which are the same or different selected from the group consisting of halogen, alkoxy, cyano, alkoxycarbonyl, amino, alkylamino and dialkylamino;
or R⁵ and R⁶, together with the nitrogen atom to which they are attached, form a saturated or unsaturated heterocyclic ring having from four to six ring atoms, which ring optionally has another ring heteroatom selected from the group consisting of nitrogen, oxygen and sulfur and is optionally substituted by from one to four substituents which are the same or different selected from the group consisting of alkyl, phenyl and benzyl;
R⁷ represents hydrogen; straight- or branched- chain alkyl having from one to six carbon atoms; or phenyl optionally substituted by from one to five substituents which are the same or different selected from the group consisting of halogen and alkoxy;
p is zero, one or two;
m is an integer from two to four;
or a pharmaceutically acceptable salt or solvate thereof.

18. A method for treating or preventing HCV in a patient in need of same comprising administering to said patient an effective anti-HCV amount of a compound of the formula (I) as defined in claim 7, or a pharmaceutically acceptable salt or solvate thereof.

19. A method inhibiting cyclophilin in a cell comprising the administration to said cell of an effective amount of a compound of formula (I) as defined in claim 1, or a pharmaceutically acceptable salt or solvate thereof.

20. A method inhibiting cyclophilin in a cell comprising the administration to said cell of an effective amount of a compound of formula (I) as defined in claim 7, or a pharmaceutically acceptable salt or solvate thereof.

21. A process for the preparation of a compound of formula (I) as defined in claim 1, comprising:
(a) reacting a compound of formula (II): 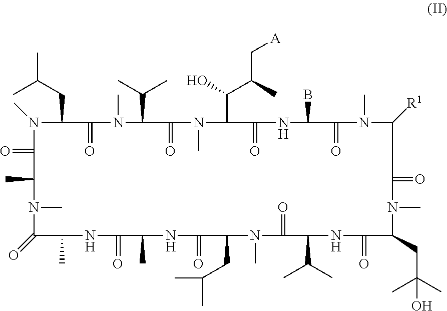
wherein A, B and R¹ are as defined in claim 1, with a compound of formula CH(OR²)₃ in the presence of a compound of formula R²—OH, wherein R² is as defined in claim 1;
(b) reacting a compound of formula (II) as defined above with a salt comprising a cation of formula (III): 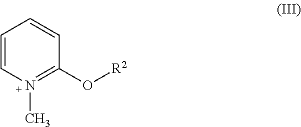
wherein R² is as defined above.

22. A process for the preparation of a compound of formula (I): 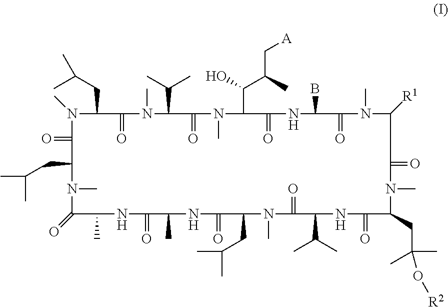
A and R² as defined in claim 1 and R¹ represents —XR³, wherein X and R³ are as defined in claim 1, comprising the treatment of the corresponding compound of formula (I) in which A and R² are as defined in claim 1 and R¹ represents hydrogen, with a base in an appropriate solvent to generate a polyanionic species, followed by the reaction of the polyanion thus obtained with a electrophile of formula R³X-L, wherein R³ and X are as defined in claim 1 and L is a leaving group.
